(19) United States
(12) Patent Application Publication
(10) Pub. No.: US 2002/0198566 A1
(43) Pub. Date: Dec. 26, 2002

(54) Device for treating living cells in a given medium

(76) Inventor: Michel Siros, Antony (FR)

Correspondence Address: ROSS J. OEHLER, AVENTIS PHARMACEUTICALS INC., ROUTE 202-206, MAIL CODE: D-303A, BRIDGEWATER, PA, 08807, US

(21) Appl. No.: 10/207,506

(22) Filed: Jul. 29, 2002

Related U.S. Application Data
(63) Continuation of application No. 09/582,720, filed on Nov. 1, 2000.
Application No. 09/582,720 is a 371 of international application No. PCT/FR99/00002, filed on Jan. 5, 1999.

(30) Foreign Application Priority Data
Jan. 6, 1998 (FR) FR98-00028

(51) Int. Cl. A61N 1/00

(52) U.S. Cl. 607/2

(57) ABSTRACT

The invention concerns a device for assisting living cell treatment in a given medium, characterised in that is comprises means (20-1) for generating an electric field (21) with substantially planar and parallel equipotential surfaces (22) and means (20-2) for applying said electric field (21) to at least part of the living cells, said means including at least two sets (23, 24) of electrode means capable of penetrating into the given medium up to the level of the living cells to be treated and means (25) for applying at least one electrical pulse between the two sets (23, 24) of electrode means. The invention is particularly advantageous for electrochemotheraphy of cancer tumours or the like, and also in laboratory for treating cell culture, liposomes and the like.

The present invention relates to devices for assisting in treating living cells in a given medium by applying electrical pulses in combination with the action of medicinal substances, this technique being known as electrochemotherapy, these devices finding particularly advantageous applications in the treatment of cancer tumours or the like, as well as in the laboratory in the treatment of cell, liposome, etc. cultures.

Specifically, it has been found that a certain number of active medicinal substances have difficulty in crossing the plasma membrane of the cells for which they are intended. To overcome this difficulty, it is consequently necessary, for example, to increase the doses of these substances, which is often difficult and almost always results in harmful side effects.

It has also been demonstrated that the application of electrical pulses to biological cells creates an at least temporary permeability of their plasma membrane, thus allowing the medicinal substances administered, for example to a patient to treat a tumour, to reach the core or nucleus of the cells and thus very considerably increase the chances of success of the treatment.

The electric fields required for carrying out electrochemotherapy were hitherto obtained in particular by applying pulses between two electrodes implanted in the tumour to be treated. An example of a device for carrying out the process described above is described in French patent No. 93/03688.

The device described in that document relates to an electrical-pulse applicator for treating biological tissues, allowing the application of an electric field to the cells of the biological tissues so as to modify the properties of their plasma membrane. This applicator comprises electrodes and a pulse generator. It comprises at least three needles. designed to be introduced into the tissues to be treated and to define a treatment volume, arranged at the apices of an equilateral triangle and forming pairs of needles, and a needle commutator which addresses the pulses produced by the pulse generator successively to the various pairs of needles.

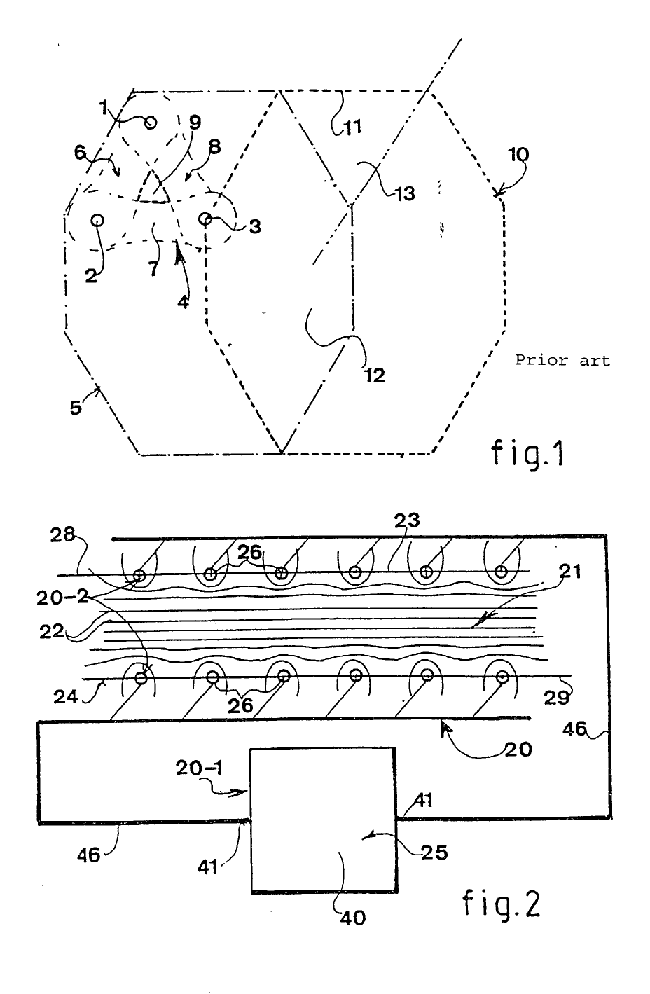

This device according to the prior art is represented schematically in FIG. 1 of the drawings attached to the description below, in which references 1 to 3 represent the three needles of a unit module 4 forming the base of the device. It is clearly understood that the device in reality comprises a plurality of these unit modules which combine with each other to form a treatment volume 5 which, on account of the shape of the unit modules 4, takes the general shape of a cylindrical volume of substantially hexagonal transverse cross section.

Such a device gives good results, but still has too many drawbacks, in particular the following four drawbacks:

since the device comprises modules of three needles 1, 2 and 3 and since the electrical pulses are applied successively to the various pairs of needles, the electric fields 6, 7 and 8 created between the pairs of needles leave a zone 9 which is not treated. It is quite clearly possible to reduce this zone, in particular by increasing the value of the electric tensions applied to the pairs of needles. However, in this case, electric tension values may be reached which induce effects, such as burns or the like, which cannot be tolerated by the patient,

the number of pairs of needles to treat a given volume is relatively large, and thus so too is the number of electrical pulses which must be applied successively since, in fact, it is equal to the number of pairs of needles possible. For example, seven needles involve the emission of twelve successive pulses, nineteen needles involve the emission of forty-two pulses, and so on,

as mentioned above, on account of the shape of the unit modules 4, the volume 5 treated necessarily takes the shape of a cylinder of substantially hexagonal transverse cross section. Two of these cross sections were represented in FIG. 1, in this instance the cross sections 10 (dashed-line curve) and 5 (dotted-line curve). However, on account of the number of needles required for the treatment, this volume cannot be too large in size and, for example, in order to treat the entire volume of a tumour, it is generally necessary to move all of the applicator needles into the tumour, gradually as a portion of the tumour corresponding to a volume 5 is treated. Furthermore, in order to be certain that no part of the tumour has been left untreated (in addition to zone 9), it is absolutely essential to move the set of applicator needles only over a distance at most equal to the length of one side 11 of the hexagonal cross section. FIG. 1 represents two successive positions 5 and 10 of the applicator and illustrates the fact that, in order to be able to treat certain parts of the tumour, for example the small volume of triangular cross section 13, it is necessary to retreat parts which have already been treated, in this instance the volume 12. It is thus apparent that the total duration of the treatment is very long and that the treatment is a greater inconvenience and more traumatizing for the patient than it should be,

finally, since the electrical pulses are only applied on two needles, the electric field generated between these two needles is only uniform in a very small part of the inter-needle space. Thus, in order to compensate for this inhomogeneity of the electric field and in order to be certain that the value of the electric field applied to the cells reaches the minimum threshold capable of creating the permeability of their plasma membrane, the electric tension generally applied between two needles is higher than it should be given the distance separating these two needles. This also results in risks of adverse effects such as burns or the like on the tissues of the patient who has to undergo a treatment, these adverse effects being very serious since they take place deep down.

Thus, one object of the present invention is to produce a device for assisting in treating living cells in a given medium, which overcomes most of the drawbacks mentioned above which are displayed in particular by the device described in the abovementioned French patent.

More specifically, a subject of the present invention is a device for assisting in treating living cells in a given medium, characterized in that it comprises means for creating an electric field having substantially planar and parallel equipotential surfaces, and means for applying this electric field to at least part of the living cells to be treated located in the said given medium.

According to another characteristic of the present invention, the means for creating an electric field having substantially planar and parallel equipotential surfaces and the means for applying this electric field to at least part of the living cells to be treated located in the said given medium comprise at least two sets of electrode means which are capable of penetrating into the given medium up to the level of the part of the living cells to be treated, and means for applying an electric tension between the two sets of electrode means.

Other characteristics and advantages of the present invention will become apparent in the course of the description which follows, given with regard to the drawings attached for illustrative but in no way limiting purposes, in which:

FIG. 1 represents a scheme of a device according to the prior art which has been described in the preamble of the present description,

FIG. 2 represents, in schematic form, one embodiment of a device according to the invention, for assisting in treating living cells in a given medium,

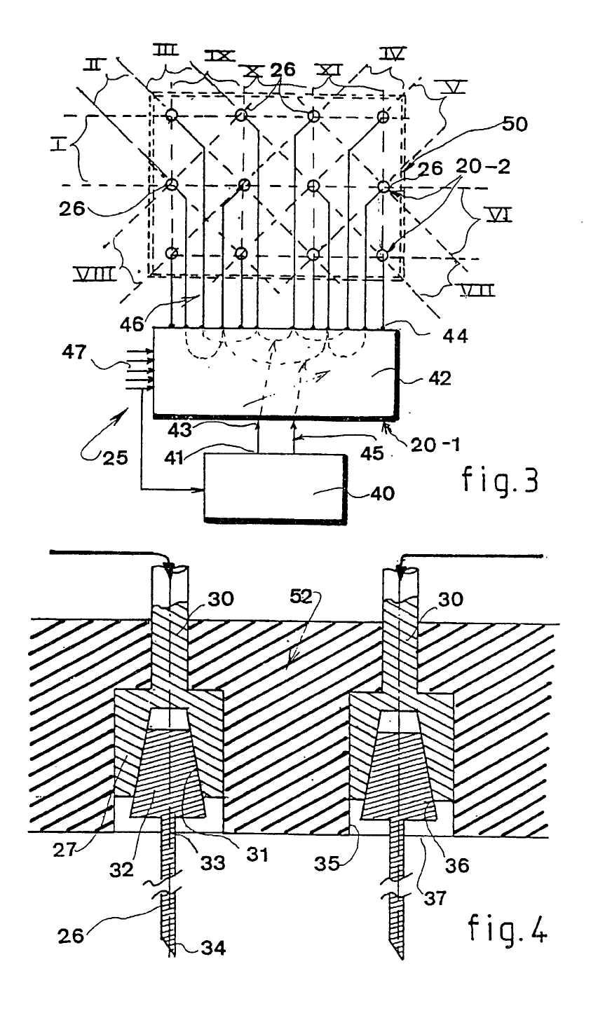

FIG. 3 represents, in schematic form, another embodiment of a device according to the invention, in accordance with the embodiment in FIG. 2, and

FIG. 4 represents a cutaway view of a detail of a device according to the invention in accordance with the embodiments illustrated in FIGS. 2 and 3.

Although the three FIGS. 2 to 4 represent different embodiments of the device according to the invention, the same reference numerals therein denote the same components, irrespective of the representation of these components and irrespective of the figure on which they are represented.

The Applicant also wishes to point out that FIGS. 2 to 4 represent different embodiments of the subject according to the invention, but that other embodiments which satisfy the definition of this invention can exist.

It is also pointed out that when, according to the definition of the invention, the subject of the invention comprises “at least one” component having a given function, the embodiment described can comprise several of these components.

It is also pointed out that, although the embodiment of the subject according to the invention as illustrated comprises several components of identical function and although, in the description, it is not specified that the subject according to this invention should necessarily comprise a particular number of these components, the subject of the invention may be defined as comprising “at least one” of these components.

It is first pointed out that the description below refers only to the three FIGS. 2 to 4, since FIG. 1 relates to the prior art cited in the preamble and has been described and commented upon hereinabove.

FIGS. 2 to 4 represent, in a schematic form, a device for assisting in treating living cells in a given medium. By way of non-limiting example, in the following description, the living cells to be treated will be those of a cancer tumour located in the flesh of a patient who in this case constitutes the given medium, but it is clearly understood that the invention is not limited to this application alone.

This device comprises means 20-1 for creating an electric field 21 having substantially planar and parallel equipotential surfaces 22, and means 20-2 for applying this electric field 21 to at least part of the tumour (FIG. 2), so as to obtain a uniform electric field in the tumour.

In one preferred embodiment, the means 20-1 for creating an electric field 21 having substantially planar and parallel equipotential surfaces 22 and the means 20-2 for applying this electric field 21 to at least part of the tumour comprise at least two sets 23, 24 of electrode means which are capable of penetrating into the flesh of the patient up to the level of the part of tumour, and means 25 for applying an electric tension between the two sets 23, 24 of electrode means.

It is clearly specified that “electric tension” means any electrical signal defined in volts or amperes, which is direct, alternating or in the form of pulses of any shape or duration.

Advantageously, each set 23, 24 of electrode means which are capable of penetrating into the flesh of the patient up to the level of the part of tumour to be treated consists of a support 52 (FIG. 4) made of an electrically insulating material, a plurality of needles 26 made of an electrically conductive material, each needle comprising a pointed end 34, and means 27 for mounting each needle 26 onto the support 52 such that they are parallel to each other and in the same plane 28, 29. Furthermore, the two planes 28, 29 in which are arranged the two pluralities of needles 26 corresponding respectively to the two sets 23, 24 are substantially parallel and the needles of the same set 23, 24 are located at equal distance to each other.

It is quite clear that, in one practical embodiment, the support 52 is the same for the two sets of needles 23, 24.

Also in a practical embodiment, the device will comprise a plurality of pairs of sets of needles 23, 24. In the embodiment illustrated in FIG. 3, it comprises 12 needles 26 which constitute eleven pairs of sets of needles 23, 24 referenced I to XI, but only one support 52 for all the needles.

To facilitate the present description, the portion of device consisting of the support 52 and all the needles solidly attached to this support will be referred to hereinbelow by the term “applicator” denoted by the reference 50.

Depending on the shape and size of the tumour to be treated, the needles 26 of the applicator 50 can have substantially the same length or different lengths.

In one advantageous embodiment illustrated in FIG. 4, the means 27 for mounting each needle 26 onto the support 52 comprise a seating 30 comprising a hollow open housing, 31. This seating is mounted, for example by plugging, in a hole 35 made in the support 52 and the means 27 also comprise a socket 32 which is solidly attached to the needle 26 at its end 33 which is opposite the pointed end 34. The socket 32 constitutes the male part of a male-female connection which is complementary to the female part consisting of the hollow housing 31. Needless to say, for an industrially advantageous embodiment and for good performance of the needles on the support 52, this male-female connection is advantageously of the conical type.

In order to be able to apply an electric tension to the needle more easily, the seating 30 and the socket 32 are made of an electrically conductive material, and one end of the seating emerges from the support 52 and can constitute an electrical connector capable of receiving an electrical conductor such as those defined below bearing the reference numeral 46 in FIGS. 2 and 3.

In addition, each seating 30 is arranged in the corresponding hole 35 such that the entry 36 of the hollow housing 31 is recessed relative to the entry 37 of the hole 35 facing the pointed end 34 of the needle. In this way, the electrical path between two proximal sockets (FIG. 4) belonging respectively to two sets of a pair 23, 24 is fairly large. This configuration avoids short circuits between these two sockets, even when they are subjected to high electrical potential differences, for example of the order of 600 volts in the case of the treatments mentioned in the preamble to the present description.

When the means 27 for mounting each needle 26 on the support 52 are made up as described above, the device also advantageously comprises means for automatically releasing the sockets 32, and thus the needles 26, from the hollow housings 31, in order, for example, to spare somebody who has to handle the applicator from accidentally injecting themselves when the treatment is complete.

These means have not been illustrated since they can be of any type, for example consisting of plates mounted slideably relative to each other such that, when they are placed in a given position, they are capable of exerting traction forces between the seatings 30 and the sockets 32 fitted with the needles 26, in order to detach the two parts of the fitting, and so that they can then free the sockets to allow the needles to fall into a container without them having to be handled by the person administering the treatment.

The process can be performed as follows to place the needles of the applicator 50 on the support 52. The needles are prearranged in a matrix on an auxiliary support, in relative positions identical to those of the hollow housings 31 on the support 52 and covered with a protective envelope. The sockets 32 are then plugged into the seatings 30, after which the envelope protecting the needles is removed.

As regards the means 25 for applying an electric tension between the two sets 23, 24 of electrode means 26 according to the embodiment illustrated in FIG. 2, they comprise an electrical power source 40 whose two outputs 41 are connected, by means of conductors 46, to the electrodes 26 of the two sets 23, 24, respectively.

When the device comprises a plurality of needles allowing the production of more than two sets of electrodes, 23, 24, as in the embodiment according to FIG. 3, the means 25 for applying an electric tension to the needles comprise an electrical power source 40 comprising electrical power supply outputs 41 and a commutator 42 which can be controlled using a control input 47, for example an electronic commutator which is well known per se. This commutator 42 is controlled, for example, by means of a computer or the like which can also control the electrical power supply 40 in order to synchronize the control of the commutator and the emission of the pulses by this source.

The electrical power source 40 is, for example, of the type described in the French patent cited in the preamble.

The commutator comprises supply inputs 43 and outputs 44, the latter advantageously being at least equal in number to the number of seatings 30 in the applicator 50, means 45 for connecting the outputs 41 of the electrical power source 40 to the supply inputs 43 of the commutator 42, and means 46 such as conductive wires for connecting each output 44 of the commutator to a seating 30.

It is thus possible to control the commutator 42 in order successively to produce the pairs of sets of needles “I” to “XI”and, each time that a pair is produced, to control the electrical power source 40 such that it emits a predetermined electrical pulse.

Incidentally, it is clearly understood that the seatings 30, the sockets 32 and the needles 26 can comprise a central channel passing through them in order to convey medicinal substances into the part of the given medium to be treated.

The device according to the invention unquestionably has advantages over the devices of the prior art, more particularly over the device described in the document cited in the preamble.

Firstly, it is found that, with the device according to the invention, the treatment requires the emission of a considerably smaller number of pulses than that which is required with the device of the prior art. If it is considered, for example, that the device according to the invention and the one according to the prior art comprise the same number of needles, for example 12 as illustrated in FIG. 3, the treatment of the part of tumour in which these 12 needles are implanted requires the application of at least 21 pulses with the device of the prior art, whereas no more than 11 pulses are required with the device according to the invention, and it is theoretically possible to treat the entire volume delimited by the 12 needles with only the two pairs of sets of needles I and VI.

It should also be mentioned that, as explained in the preamble, with the device of the prior art there nevertheless remains uncertainty regarding the quality of the treatment of the prismatic volumes defined, such as the volume bearing the reference numeral 9 in FIG. 1. With the device according to the invention, there is no risk of there being an untreated zone in the part of tumour in which the needles are implanted.

In the device according to the invention, it is found that the needles are arranged in two ways relative to each other, in a “square” for the pairs of sets I, VI, IX, X and XI, and in “staggered rows” for the pairs II, III, IV, V, VII and VIII, but they delimit a rectangular parallelepipedal treatment volume, thus making it possible to treat the entire tumour by stepwise movement of the applicator 50 without it being necessary, during the new implantation of the needles, to recover a part of the previous volume treated, as in the case of the treatment with the device of the prior art to treat the zones bearing the reference numeral 13 in FIG. 1.

The device according to the invention thus allows faster treatments than that of the prior art and with better efficacy, and thus in conclusion less inconvenient and traumatizing treatments for the patients.

1. Device for assisting in treating living cells in a given medium, characterized in that it comprises:
means (20-1) for creating an electric field (21) having substantially planar and parallel equipotential surfaces (22), and
means (20-2) for applying this electric field (21) to at least part of the living cells to be treated located in the given medium.

2. Device according to claim 1, characterized in that the means (20-1) for creating an electric field (21) having substantially planar and parallel equipotential surfaces (22) and the means (20-2) for applying this electric field (21) to at least part of the living cells to be treated located in the given medium comprise:
at least two sets (23, 24) of electrode means which are capable of penetrating into the given medium up to the level of the part of the living cells to be treated, and
means (25) for applying an electric tension between the two sets (23, 24) of electrode means.

3. Device according to claim 2, characterized in that each set (23, 24) of electrode means which are capable of penetrating into the given medium up to the level of the part of the living cells to be treated consists of:
a support (52) made of an electrically insulating material,
a plurality of needles (26) made of an electrically conductive material, each needle comprising a pointed end (34), and
means (27) for mounting each needle (26) onto the support (52) such that they are parallel to each other and in the same plane (28, 29).

4. Device according to claim 3, characterized in that the two planes (28, 29) in which are arranged the two pluralities of needles (26) are substantially parallel, and that the needles (26) of the same set (23, 24) are located at equal distance to each other.

5. Device according to either of claims 3 and 4, characterized in that the means (27) for mounting each needle (26) onto the support (52) consist of:
a seating (30), the said seating comprising a hollow housing (31) and being mounted in a hole (35) made in the said support (52), and
a socket (32) which is solidly attached to the needle (26) at its end (33) which is opposite the pointed end (34), the said socket (32) constituting the male part of a male-female connection which is complementary to the female part consisting of the said hollow housing (31).

6. Device according to claim 5, characterized in that the male-female connection is of the conical type.

7. Device according to either of claims 5 and 6, characterized in that the said seating (30) is arranged in the said hole such that the entry (36) of the hollow housing (31) is recessed relative to the entry (37) of the said hole (35) facing the pointed end (34) of the needle.

8. Device according to one of claims 5 to 7, characterized in that the means (25) for applying an electric tension between the two sets (23, 24) of electrode means comprise at least one electrical power source (40) comprising electrical power supply outputs (41), a commutator (42) which can be controlled using a control input (47), the said commutator comprising supply inputs (43) and outputs (44), these outputs (44) being at least equal in number to the number of seatings (30), means (45) for connecting the outputs (41) of the said electrical power source (40) to the supply inputs (43) of the said commutator (42), and means (46) for connecting each output (44) of the commutator (42) to a seating (30), respectively.

9. Device according to one of claims 5 to 8, characterized in that the said seating (30) and the said socket (32) are made of an electrically conductive material.

10. Device according to one of claims 5 to 9, characterized in that it comprises means for automatically releasing the socket (32) from the hollow housing (31).
